(19) United States
(12) Patent Application Publication
(10) Pub. No.: US 2006/0247271 A1
(43) Pub. Date: Nov. 2, 2006

(54) Sulphonyl hydroxamic acid derivatives as inhibitors of s-cd23

(76) Inventor: Gordon Bruton, Harlow, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/543,974

(22) PCT Filed: Jan. 30, 2004
(86) PCT No.: PCT/EP04/00954

§ 371 Date: Mar. 10, 2006

(30) Foreign Application Priority Data
Jan. 30, 2003 (GB) 0302431.2

(51) Int. Cl. A61K 31/47; C07D 215/12; A61K 31/19

(52) U.S. Cl. 514/311; 546/176; 514/575

(57) ABSTRACT

Compounds of formula (I): 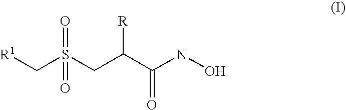
wherein R is hydrogen, alkyl, alkoxy, alkenyl, alkynyl, aryl, heteroaryl or heterocyclyl; and R¹ is bicyclyl or heterobicyclyl; are useful in the treatment and prophylaxis of conditions mediated by s-CD23.

This invention relates to novel inhibitors of the formation of soluble human CD23 and their use in the treatment of conditions associated with excess production of soluble CD23 (s-CD23) such as autoimmune disease and allergy.

CD23 (the low affinity IgE receptor FceRII, Blast 2), is a 45 kDa type II integral protein expressed on the surface of a variety of mature cells, including B and T lymphocytes, macrophages, natural killer cells, Langerhans cells, monocytes and platelets (Delespesse et al, Adv Immunol, 49 [1991] 149-191). There is also a CD23-like molecule on eosinophils (Grangette et al, J Immunol, 143 [1989] 3580.3588). CD23 has been implicated in the regulation of the immune response (Delespesse et al, Immunol Rev, 125 [1992] 77-97). Human CD23 exists as two differentially regulated isoforms, a and b, which differ only in the amino acids at the intracellular N-terminus (Yokota et al, Cell, 55 [1988] 611-618). In man the constitutive a isoform is found only on B-lymphocytes, whereas type b, inducible by IL4, is found on all cells capable of expressing CD23.

Intact, cell bound CD23 (i-CD23) is known to undergo cleavage from the cell surface leading to the formation of a number of well-defined soluble fragments (s-CD23), which are produced as a result of a complex sequence of proteolytic events, the mechanism of which is still poorly understood (Bourget et al J Biol Chem, 269 [1994] 6927-6930). Although not yet proven, it is postulated that the major soluble fragments (Mr 37, 33, 29 and 25 kDa) of these proteolytic events, all of which retain the C-terminal lectin domain common to i-CD23, occur sequentially via initial formation of the 37 kDa fragment (Letellier et al, J Exp Med, 172 [1990] 693-700). An alternative intracellular cleavage pathway leads to a stable 16 kDa fragment differing in the C-terminal domain from i-CD23 (Grenier-Brosette et al, Eur J Immunol, 22 [1992] 1573-1577).

Several activities have been ascribed to membrane bound i-CD23 in humans, all of which have been shown to play a role in IgE regulation. Particular activities include: a) antigen presentation, b) IgE mediated eosinophil cytotoxicity, c) B cell homing to germinal centres of lymph nodes and spleen, and d) downregulation of IgE synthesis (Delespesse et al, Adv Immunol, 49, [1991] 149-191). The three higher molecular weight soluble CD23 fragments (Mr 37, 33 and 29 kDa) have multifunctional cytokine properties which appear to play a major role in IgE production. Thus, the excessive formation of s-CD23 has been implicated in the overproduction of IgE, the hallmark of allergic diseases such as extrinsic asthma, rhinitis, allergic conjunctivitis, eczema, atopic dermatitis and anaphylaxis (Sutton and Gould, Nature, 3, [1993] 421-428).

Other biological activities attributed to s-CD23 include the stimulation of B cell growth and the induction of the release of mediators from monocytes. Thus, elevated levels of s-CD23 have been observed in the serum of patients having B-chronic lymphocytic leukaemia (Sarfati et al, Blood, 71 [1988]94-98) and in the synovial fluids of patients with rheumatoid arthritis (Chomarat et al, Arthritis and Rheumatism, 36 [1993] 234-242). That there is a role for CD23 in inflammation is suggested by a number of sources. First, sCD23 has been reported to bind to extracellular receptors which when activated are involved in cell-mediated events of inflammation. Thus, sCD23 is reported to directly activate monocyte TNF, IL-1, and IL-6 release (Armant et al, vol 180, J.Exp. Med., 1005-1011 (1994)). CD23 has been reported to interact with the B2-integrin adhesion molecules, CD11b and CD11c on monocyte/macrophage (S. Lecoanet-Henchoz et al, Immunity, vol 3; 119-125 (1995)) which trigger NO2⁻, hydrogen peroxide and cytoline (IL-1, IL-6, and TNF) release. Finally, IL-4 or IFN induce the expression of CD23 and its release as sCD23 by human monocytes. Ligation of the membrane bound CD23 receptor with IgE/anti-IgE immune complexes or anti CD23 mAb activates cAMP and IL-6 production and thromboxane B2 formation, demonstrating a receptor-mediated role of CD23 in inflammation.

Because of these various properties of CD23, compounds which inhibit the formation of s-CD23 should have twofold actions of a) enhancing negative feedback inhibition of IgE synthesis by maintaining levels of i-CD23 on the surface of B cells, and b) inhibiting the immunostimulatory cytokine activities of higher molecular weight soluble fragments (Mr 37, 33 and 29 kDa) of s-CD23. In addition, inhibition of CD23 cleavage should mitigate sCD23-induced monocyte activation and mediator formation, thereby reducing the inflammatory response.

International Patent Application No. WO 97/27174 (Shionogi & Co., Ltd) and International Patent Application number WO 95/35275 (British Biotech Ltd) disclose that certain compounds of formula (A): 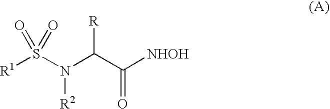
wherein R¹ is arylalkyl or heteroarylalkyl are effective inhibitors of metalloproteinases.

International Patent Application No. WO 98/46563 (British Biotech Ltd) discloses that certain compounds of formula (A) above in which R¹ is phenylalkyl or heteroarylalkyl are effective inhibitors of matrix metalloproteases.

WO 99/06361 (Abbott) and WO 00/12478 (Zeneca Limited) describe a range of compounds which includes reverse hydroxamate sulfonyl and sulfonamide compounds, for use as metalloproteinase inhibitors.

WO 99/38843 (Darwin Discovery Limited) discloses a generic scope of compounds useful in the treatment of inter alia conditions mediated by enzymes involved in the shedding of CD23, which covers compounds of the formula (B): 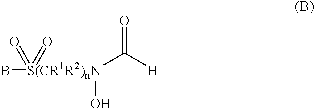
wherein B, R¹ and R² are selected from a range of organic groups.

PCT EP01/05798 discloses compounds useful in the treatment and prophylaxis of conditions mediated by enzymes involved in the shedding of CD23 which covers compounds of formula (C): 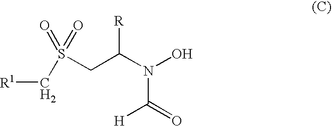
Wherein R is hydrogen, alkyl, alkenyl, alkynyl, aryl, heteroaryl or heterocyclyl; and R¹ is bicyclyl or heterobicyclyl.

According to the present invention, there is provided a compound of formula (I): 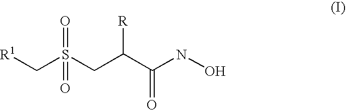
wherein

R is hydrogen, alkyl, alkenyl, alkynyl, alkoxy, aryl, heteroaryl or heterocyclyl; and

R¹ is bicyclyl or heterobicyclyl;

Alkyl, alkoxy, alkenyl and alkynyl groups referred to herein either alone or as part of another group may be straight, branched or cyclic.

Alkyl, alkoxy, alkenyl and alkynyl groups referred to herein in the definition of the R group contain up to eight carbon atoms and are optionally substituted by one or more groups selected from the group consisting of aryl, heterocyclyl, (C1-6)alkylthio, (C2-6)alkenylthio, (C2-6)alkynylthio, aryloxy, arylthio, heterocyclyloxy, heterocyclylthio, (C1-6)alkoxy, aryl(C1-6)alkoxy, aryl(C1-6)alkylthio, amino, mono- or di-(C1-6)alkylamino, acylamino and sulfonylamino in which the amino group may optionally be substituted by (C1-6)alkyl, cycloalkyl, cycloalkenyl, carboxylic acid (C1-6) esters, hydroxy, halogen and carboxamide: CONR²R³ where R² and R³ are independently selected from the group consisting of hydrogen, alkyl, aryl, arylalkyl and heterocyclyl, and includes R² and R³ as part of a heterocyclyl group.

Cycloalkyl and cycloalkenyl groups referred to herein in the definition of the R group include groups having between three and eight ring carbon atoms and are optionally substituted as described hereinabove for alkyl, alkenyl and alkynyl groups.

When used herein in the definition of the R group the term “aryl” includes phenyl. Suitably any aryl group, including phenyl, may be optionally substituted by up to five, preferably up to three substituents. Suitably, any two substituents may optionally together form a fused ring and may optionally be interrupted by up to three heteroatoms in the ring, each of which is selected from oxygen, nitrogen and sulphur. Suitable substituents include halogen, halo(C1-6)alkyl or polyhalo(C1-6)alkyl e.g. CF₃, halo(C1-6)alkyloxy or polyhalo(C1-6)alkyloxy, e.g. OCF₃, CN, (C₁₋₆)alkyl, (C₁₋₆)alkoxy, hydroxy, amino, mono- and di-N—(C1-6)alkylamino, acylamino (e.g. acetylamino) in which the amino group may optionally be substituted by (C1-6)alkyl, acyloxy, carboxy, (C1-6)alkoxycarbonyl, aminocarbonyl, mono- and di-N—(C1-6)akyleaninocarbonyl, mono- and di-N—(C1-6)alkylaminoalkyl, (C1-6)alkylsulfonylamino in which the amino group may optionally be substituted by (C1-6)alkyl, aryl(C1-6)alkoxycarbonylamino in which the amino group may optionally be substituted by (C1-6)alkyl, (C1-6)alkoxycarbonylamino in which the amino group may optionally be substituted by (C1-6)alkyl, aminosulfonyl, (C1-6)alkylthio, (C1-6)alkylsulfonyl, (C1-6)alkylsulfonyloxy, mono- and di-N—(C1-6)alkylaminosulfonyl, heterocyclyl, heterocyclyl(C1-6)alkyl, aminosulfonyloxy and (C1-6)mono- and dialkylaminosulfonyloxy. The term “aryl” includes single and fused rings, of which at least one is aromatic, which rings may be unsubstituted or substituted by, for example, up to three substituents as set out above. Each ring suitably has from 4 to 7, preferably 6 or 7, ring atoms.

When used herein in the definition of the R group the term “heteroaryl” suitably includes any heterocyclyl group which incorporates at least one aromatic ring (heterocyclic or carbocyclic). Suitable heteroaryl groups include thiophene, such as thiophen-2-yl and thiophen-3-yl; furan, such as furan-2-yl and furan-3-yl; benzothiophene, such as benzothiophen-2-yl; pyrazole, such as pyrazol-3-yl; and isoxazole, such as isoxazol-3-yl.

When used herein in the definition of the R group the terms “heterocyclyl” and “heterocyclic” suitably include, unless otherwise defined, aromatic and non-aromatic, single and fused, rings, one or more rings suitably containing up to four heteroatoms in each ring, each of which is selected from oxygen, nitrogen and sulphur, which rings, may be unsubstituted or substituted by, for example, up to three substituents. Each ring suitably has from 4 to 7, preferably 5 or 6, ring atoms. A fused heterocyclic ring system may include carbocyclic rings and need include only one heterocyclic ring. Suitable heteroaryl groups include benzodioxan, such as 2,3-dihydrobenzo[1,4]dioxin-6-yl; benzodioxepine, such as 3,4-dihydro-2H-benzo[b][1,4]dioxepin-7-yl; and benzoxazine, such as 3-oxo-3,4-dihydro-2H-benz[1,4]oxazin-6-yl.

Suitable substituents for a heteroaryl or heterocyclyl group include halogen, halo(C1-6)alkyl or polyhalo(C1-6)alkyl e.g. CF₃, halo(C1-6)alkyloxy or polyhalo(C1-6)alkyloxy, e.g. OCF₃, (C1-6)alkyl, (C1-6)alkoxy, hydroxy, CN, amino, mono-and di-N—(C1-6)alkylamino, acylamino (e.g. acetylamino) in which the amino group may optionally be substituted by (C1-6)alkyl, acyloxy, carboxy, (C1-6)alkoxycarbonyl, aminocarbonyl, mono- and di-N—(C1-6)alkylaminocarbonyl, (C1-6)alkylsulfonylamino, aminosulfonyl, mono- and di-N—(C1-6)alkylaminosulfonyl, (C1-6)alkylthio and (C1-6)alkylsulfonyl.

When used herein in the definition of the R¹ group “bicyclyl” means fused bicyclic rings suitably containing 4 to 7, preferably 5 or 6 ring atoms in each ring. One ring of the bicyclyl may be saturated or partially saturated. Suitable bicyclyl groups include naphthyl such as 2-naphthyl, tetrahydronaphthyl such as 1,2,3,4-tetrahydronaphthalen-2-yl, and indanyl such as 2-indanyl.

When used herein in the definition of the R¹ group, heterobicyclyl means fused bicyclic aromatic and non-aromatic rings containing up to 4 heteroatoms in each ring, each of which is selected from oxygen, nitrogen and sulphur. Each ring suitably has from 4 to 7, preferably 5 or 6, ring atoms. The fused bicyclic ring system may include one carbocyclic ring and one of the rings may be saturated or partially saturated. Suitable heterobicyclyl groups include benzothiophene, such as benzothiophen-5-yl and benzothiophen-6-yl; benzofuran such as benzofuran-2-yl, benzofuran-5-yl and benzofuran-6-yl; quinoline such as quinolin-3-yl; thienopyridine such as thieno[2,3-b]pyridin-5-yl and thieno[3,2-b]pyridin-6-yl; isoquinoline such as isoquinolin-3-yl;. quinoxaline such as quinoxalin-2-yl; and benzothiazole such as benzothiazol-6-yl.

Aromatic rings in bicyclyl and heterobicyclyl ring systems may be optionally substituted with up to three substituents. Suitable substituents include fluorine. Examples of substituted heterobicyclyl groups include 2-fluorobenzothiophen-5-yl and 3-fluorobenzothiophen-5-yl.

In a particular aspect of the invention, R is phenyl or alkoxy, and/or R¹ is quinoline. Preferably, R is phenyl or propyloxy, an/or R¹ is quinolin-3-yl. More preferably, the compound of formula (I) of the invention is selected from the group consisting of the compounds described in the Examples hereinbelow.

According to a further aspect, the present invention provides the use of a compound of formula (I) for the production of a medicament for the treatment or prophylaxis of disorders such as allergy, allergic asthma, atopic dermatitis and other atopic diseases, inflammatory disorders, and autoimmune disease, in which the overproduction of s-CD23 is implicated.

In a further aspect the invention provides a method for the treatment or prophylaxis of disorders such as allergy, allergic asthma, atopic dermatitis and other atopic diseases, inflammatory disorders, and autoimmune disease, in which the overproduction of s-CD23 is implicated, which method comprises the administration of a compound of formula (I), to a human or non-human mammal in need thereof.

The invention also provides a pharmaceutical composition for the treatment or prophylaxis of disorders such as allergy, allergic asthma, atopic dermatitis and other atopic diseases, inflammatory disorders, and autoimmune disease, in which the overproduction of s-CD23 is implicated which comprises a compound of formula (1) and optionally a pharmaceutically acceptable carrier thereof.

Particular inflammatory disorders include CNS disorders such as Alzheimer's disease, multiple sclerosis, and multi-infarct dementia, as well as the inflammation mediated sequel of stroke and head trauma

It is to be understood that the pharmaceutically acceptable salts, solvates and other pharmaceutically acceptable derivatives of the compound of formula (I) are also included in the present invention.

Salts of compounds of formula (I) include for example acid addition salts derived from inorganic or organic acids, such as hydrochlorides, hydrobromides, hydroiodides, p-toluenesulphonates, phosphates, sulphates, acetates, trifluoroacetates, propionates, citrates, maleates, fumarates, malonates, succinates, lactates, oxalates, tartrates and benzoates.

Salts may also be formed with bases. Such salts include salts derived from inorganic or organic bases, for example alkali metal salts such as sodium or potassium salts, and organic amine salts such as morpholine, piperidine, dimethylamine or diethylamine salts.

The compounds of the invention may be prepared by use of any appropriate conventional method.

Accordingly, a further aspect of the invention provides a process for preparing a compound of formula (1) as defined hereinabove, which process comprises:

(a) deprotecting a compound of formula (II): 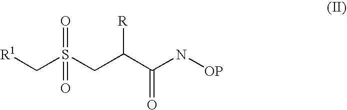
wherein R and R¹ are as defined hereinabove, and P is a protecting group such as allyl, allyloxycarbonyl, benzyl, benzyloxycarbonyl, tetrahydropyranyl, p-methoxybenzyl, t-butyldimethylsilyl or trimethylsilyl, acyl such as acetyl or benzoyl or

(b) oxidising a compound of formula (III): 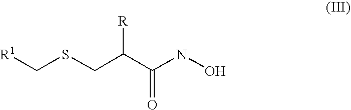
wherein R and R¹ are as defined hereinabove, or

(c) converting a compound of formula (I) to a different compound of formula (I) as defined hereinabove, or

(d) reacting a compound of formula (VIII): 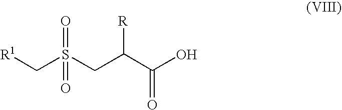
with hydroxylamine or a salt thereof.

Compounds of formula (II), (III) or (VIII) are novel and form a further aspect of the invention.

The following reaction schemes illustrate the procedures that may be used to prepare compounds of formula (I).

### Reaction Schemes

One procedure for preparing compounds of formula (I) is shown in Scheme 1. A thiol, or thiol precursor such as a thioacetate (IV), reacts with an acrylate ester such as (V), wherein Q is a protecting or leaving group to give a sulfide (VI). Oxidation of the sulfide (VI) to the sulfone (VII) can be accomplished with a per-acid, such as meta-chloroperoxybenzoic acid. Conversion of the ester (VII) to the acid (VIII) can be effected under basic or acidic hydrolytic conditions such as sodium hydroxide or HCl, by hydrogenation when the ester is hydrogenolysable such as benzyl, or by specific deprotection of standard protecting groups such as TFA for tert-butyl esters. Activation of the acid by conversion to an acid chloride, mixed anhydride or N-hydroxyheterocycle, and subsequent reaction with hydroxylamine, an in-situ protected hydroxylamine or a protected hydroxylamine with subsequent compatible deprotection, such as hydrogenolysis of an O-benzyl hydroxylamine, affords the hydroxamic acids (I)

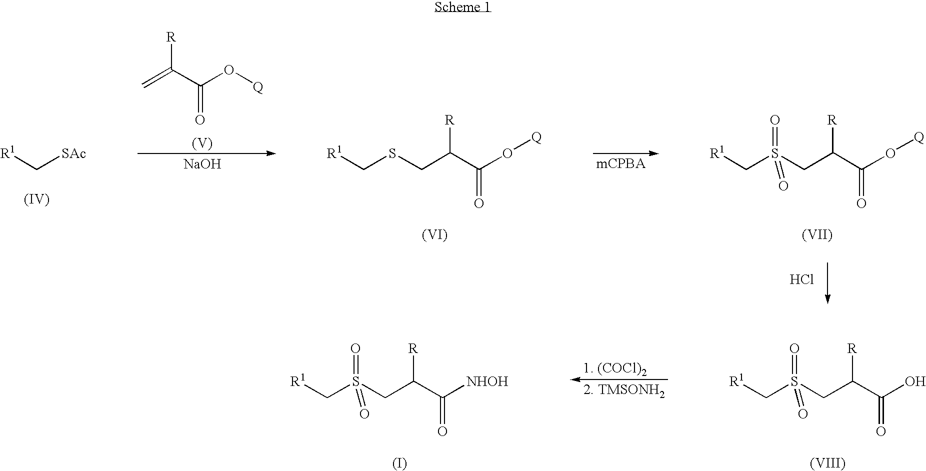

The isomers, including stereoisomers, of the compounds of the present invention may be prepared as mixtures of such isomers or as individual isomers. The individual isomers may be prepared by any appropriate method, for example individual stereoisomers may be prepared by stereospecific chemical synthesis starting from chiral substrates or by separating mixtures of enantiomers or mixtures of diasteromers using known methods such as chiral preparative HPLC.

In a preferred aspect, the present invention provides a compound of formula (IA):

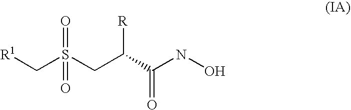

Hydroxamic acids of type (IA) are accessible, as described in Scheme 2, from chiral diols (XA) wherein P is a protecting group. These diols are readily available, including from commercially available dioxalones (IXA) wherin P is a methyl ester, after acidic deprotection. Suitable protection of the primary alcohol of (XA), with a silylhalide and amine base for example, and subsequent alkylation of (XIA), with an alkyl halide mesylate or tosylate in the presence of a base, such as sodium hydride, afords the ethers (XIIA), wherein Y is an alkyl(C₁₋₈). When alkylation of (XIA) has been achieved using an allylic halide, mesylate or tosylate then the resulting allylic ether (XIA) may be further modified by hydrogenating with hydrogen and a suitable catalyst such as palladium to give (XIIA). Deprotection of (XIIA) to the primary alcohol (XIIIA), under acidic or fluoride catalysis for removal of silyl groups, and reaction with thioacetic acid under Mitsunobu conditions gives the thioacetate (XIVA). In-situ conversion of (XIVA) to a thiol with sodium hydroxide or methoxide and alkylation of the liberated thiol with a halide affords sulfides (XVIA). Oxidation of sulfides (XVIA) with per-acids, such as meta-chloroperoxybenzoic acid, affords sulfones (XVIIA). Removal of the protecting ester group with an appropriate standard methodology or acid hydrolysis of alkyl esters provides the carboxylates (XVIA). Activation of the acid (XVIII)A, by conversion to an acid chloride, mixed anhydride or N-hydroxyheterocycle, and subsequent reaction with hydroxylamine, an in-situ protected hydroxylamine or a protected hydroxylamine with subsequent compatible deprotection, such as hydrogenolysis of an O-benzyl hydroxylamine, affords the hydroxamic acids (IA).

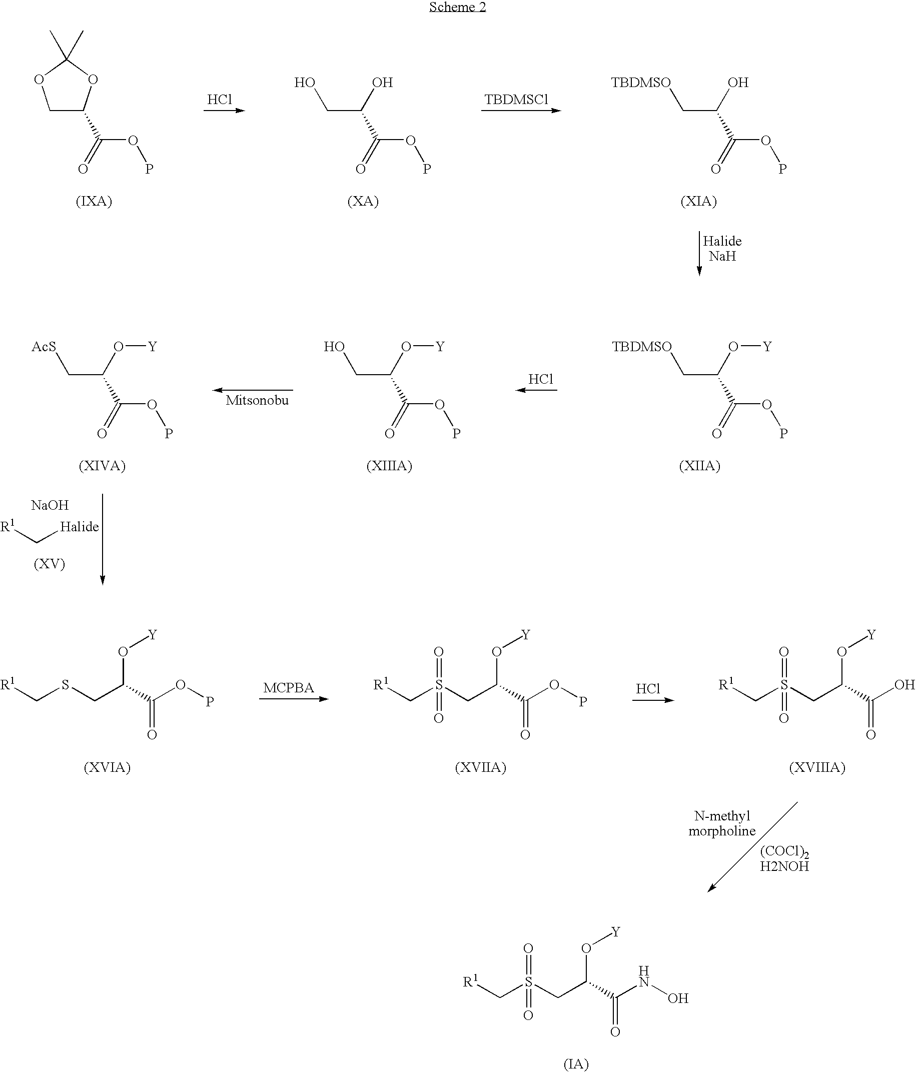

The other starting materials and other reagents are available commercially or can be synthesised by well-known and conventional methods.

It is preferred that the compounds are isolated in substantially pure form.

As stated herein an inhibitor of the formation of soluble human CD23 has useful medical properties. Preferably the active compounds are administered as pharmaceutically acceptable compositions.

The compositions are preferably adapted for oral administration. However, they may be adapted for other modes of administration, for example in the form of a spray, aerosol or other conventional method for inhalation, for treating respiratory tract disorders; or parenteral administration for patients suffering from heart failure. Other alternative modes of administration include sublingual or transdermal administration.

The compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystaline cellulose; or pharmaceutically acceptable wetting agents such as sodium lauryl sulphate.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring-agents.

For parenteral administration, fluid unit dosage forms are prepared utilising the compound and a sterile vehicle, and, depending on the concentration used, can be either suspended or dissolved in the vehicle. In preparing solutions the compound can be dissolved in water for injection and filter sterilised before filling into a suitable vial or ampoule and sealing. Advantageously, adjuvants such as a local anaesthetic, a preservative and buffering agents can be dissolved in the vehicle. To enhance the stability, the composition can be frozen after filing into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

Compositions of this invention may also suitably be presented for administration to the respiratory tract as a snuff or an aerosol or solution for a nebulizer, or as a microfine powder for insuflation, alone or in combination with an inert carrier such as lactose. In such a case the particles of active compound suitably have diameters of less than 50 microns, preferably less than 10 microns for example diameters in the range of 1-50 microns, 1-10 microns or 1-5 microns. Where appropriate, small amounts of other anti-asthmatics and bronchodilators, for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included.

The compositions may contain from 0.1% to 99% by weight, preferably from 10-60% by weight, of the active material, depending upon the method of administration. A preferred range for inhaled administration is 10-99%, especially 60-99%, for example 90, 95 or 99%.

Microfine powder formulations may suitably be administered in an aerosol as a metered dose or by means of a suitable breath-activated device.

Suitable metered dose aerosol formulations comprise conventional propellants, cosolvents, such as ethanol, surfactants such as oleyl alcohol, lubricants such as oleyl alcohol, desiccants such as calcium sulphate and density modifiers such as sodium chloride.

Suitable solutions for a nebulizer are isotonic sterilised solutions, optionally buffered, at for example between pH 47, containing up to 20 mg/ml of compound but more generally 0.1 to 10 mg/ml, for use with standard nebulisation equipment.

An effective amount will depend on the relative efficacy of the compounds of the present invention, the severity of the disorder being treated and the weight of the sufferer. Suitably, a unit dose form of a composition of the invention may contain from 0.1 to 1000 mg of a compound of the invention (0.001 to 10 mg via inhalation) and more usually from 1 to 500 mg, for example 1 to 25 or 5 to 500 mg. Such compositions may be administered from 1 to 6 times a day, more usually from 2 to 4 times a day, in a manner such that the daily dose is from 1 mg to 1 g for a 70 kg human adult and more particularly from 5 to 500 mg. That is in the range of about 1.4×10m2 mg/kg/day to 14 mg/kg/day and more particularly in the range of about 7×10-2 mg/kg/day to 7 mg/kg/day.

The following examples illustrate the invention but do not limit it in any way.

### Preparation 1: 3-Acetylthiomethylquinoline

Method A

Step 1: 3-Quinolylmethanol—Quinoline-3-carboxaldehyde (13.18 g) in ethanol (260 ml) was cooled to 0° C. followed by the addition of sodium borohydride (1.62 g) portionwise. The temperature was maintained at 0° C. for 15 min followed by the addition of 6N HCl (28 ml) during which time the temperature of the reaction was maintained between 0-5° C. The solution was then neutralised with 1M NaOH. The crude reaction mixture was stripped to dryness to remove ethanol and the residue was partitioned between water and EtOAc. The EtOAc layer was then dried (MgSO₄) and absorbed onto silica gel and chromatographed (flash silica gel, step gradient: 0-100% EtOAc/hexane) to give the subtitle compound as a white solid (9.85 g).

Step 2: 3-Chloromethylquinoline hydrochloride—3-Quinolylmethanol (9.85 g) was taken up in dry benzene (200 ml) and stirred followed by the addition of thionyl chloride (14.69 ml). An immediate yellow precipitate was obtained. Stirring was maintained at rt for 2 h. A light yellow solid was filtered off and dried to give the subtitle compound (13 g).

Step 3: 3-Acetylthiomethylquinoline—3-Chloromethylquinoline hydrochloride (5.2 g) was taken up in acetone (100 ml) followed by the addition of potassium thioacetate (1.8 g) and allowed to stir at rt overnight. The reaction mixture was absorbed onto silica gel and chromatographed (silica gel, step gradient 0-50% ether/petroleum ether) to give the title compound as an orange solid (4.2 g). ¹H NMR δ(DMSO-d6): 8.85 (1H, d, J=2 Hz), 8.25(1H, d, J=2 Hz), 8.01(1H, d, J=8.4 Hz), 7.95 (1H, d, J=8.4 Hz), 7.74 (1H, t, J=8.4 Hz), 7.61 (1H, t, J=8.4 Hz), 4.33 (2H, s), 2.38 (3H, s).

Method B

3-Methylquinoline (5 g) in CCl₄ (50 ml) was treated with glacial acetic acid (1.85 ml), NBS (8.5 g) and AIBN (1.5 g). The reaction was brought to reflux using a 100W halogen light and refluxed for 10 min. After cooling, EtOAc (60 ml) was added and the reaction was filtered through a plug of silica, concentrated to half volume and added to potassium thioacetate (10 g) dissolved in DMF (150 ml) with potassium carbonate (2 g). The reaction was then further concentrated to 150 ml by evaporation. After 2 h the reaction was diluted with EtOAc (300 ml) and washed with saturated sodium hydrogen carbonate solution and saturated brine (8×). The organic phase was evaporated and the residue chromatographed (silica gel, step gradient 0-50% ether/petroleum ether) to afford the title compound (3.1 g).

### EXAMPLE 1

### N-Hydroxy-2-phenyl-3-(3-quinolylmethanesulfonyl)-propionamide

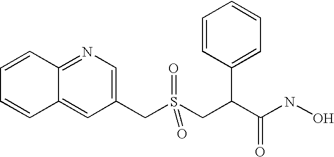

Step 1: Ethyl 2-phenyl-3-(3-quinolylmethanesulfanyl)-propanoate—To a solution of 3-acetylthiomethylquinoline (0.65 g) in ethanol (6 ml) was added aqueous sodium hydroxide (1.5 ml, 2M). After 10 min a solution of ethyl 1-phenylpropenoate (J R Ames and W Davey, J Chem Soc, 1958, 1794) (0.62 g) in ethanol (2 ml) was added and after stirring for 30 min the solution was partitioned between aqueous ammonium chloride and diethyl ether. The organic layer was dried (MgSO₄) and evaporated and the residue chromatographed on silica eluting with hexane—ethyl acetate mixtures to give the subtitle compound (0:64 g).

Step 2: Ethyl 2-phenyl-3-(3-quinolylmethanesulfonylpropanoate—A solution of ethyl 2-phenyl-3-(3-quinolylmethanesulfanyl)-propanoate (0.64 g) in dichloromethane (10 ml) at 0° C. was treated with 3-chloroperoxybenzoic acid (65%, 900 mg) and after stirring for 30 min the solution was partitioned between aqueous sodium hydrogen carbonate containing 1% sodium sulfite and dichloromethane. The organic layer was dried (MgSO₄) and evaporated and the residue chromatographed on silica eluting with hexane—ethyl acetate mixtures to give the subtitle compound (0.40 g).

Step 3: 2-Phenyl-3-(3-quinolylmethanesulfonyl)-propanoic acid hydrochloride—A solution of ethyl 2-phenyl-3-(3-quinolylmethanesulfonyl)-propanoate (0.30 g) in hydrochloric acid (5 ml, 11M) was heated at reflux for 2 h then evaporated to give the subtitle compound (0.27 g).

Step 4: N-Hydroxy-2-phenyl-3-(3-qinolylmethanesulfonyl)-propionamide—A suspension of 2-phenyl-3-(3-quinolylmethanesulfonyl)-propanoic acid hydrochloride (30 mg) in dichloromethane (5 ml) was treated with oxalyl chloride (0.5 ml) and DMF (1 drop). After 1h the mixture was evaporated and the resulting solid suspended in more dichloromethane (5 ml) and O-trimethylsilylhydroxylamine (0.5 ml) added. After 1 h water was added and the pH adjusted to 7 with hydrochloric acid (1M) then extracted with ethyl acetate. The organic extract was dried (MgSO₄) and evaporated and the residue crystallised from diethyl ether to give the title compound (14 mg). MS (+ve ion electrospray) 371 (MH⁺, 100%), ¹H NMR δ(CD₃OD) 8.9 (1H, m), 8.4 (1H, m), 8.09 (1H, d, J=8 Hz), 7.95 (1H, d, J=8 Hz), 7.78 (1H, t, J=8 Hz), 7.62 (1H, t, J=8 Hz), 7.2-7.4 (5H, m), 4.60 (2H, ABq), 4.1 and 4.2 (2H, 2m), and 3.5 (1H, m).

### EXAMPLE 2

### (R)—N-Hydroxy-2-propoxy-3-(quinolin-3-ylmethanesulfonyl)-propionamide

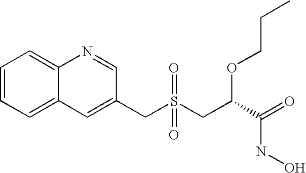

Step 1: Methyl (S)-3-(t-butyldimethylsilyloxy)-2-hydroxypropionate—A stirred solution of methyl (S)-2,2-dimethyl-1,3-dioxalane-4-carboxylate (5.67 g) in dry MeOH (90 ml) at rt was treated with 4M HCl in dioxan. After 2 h the solution was evaporated and then re-evaporated from MeOH/toluene (2×30 ml) and CHCl₃ (30 ml). The diol was dissolved in dry DMF (80 ml) and cooled in ice, The stirred solution was treated with imidazole (2.41 g) and t-butyldimethylsilyl chloride (5.34 g). After 2 h the mixture was diluted with EtOAc (500), washed with water (3×200 ml),saturated brine (100 ml), dried MgSO₄) and evaporated. The residue was cchromatographed (silica gel, stepgradient 0-15% EtOAc in Light petroleum 40°-60°) to give the subtitle compound (5.85 g). ¹H NMR δ(CDCl3) 4.18 (1H,m), 3.89 (2H,m), 3.74 (3H,s), 2.97 (1H,d,J=8 Hz), (0.83 (9H,s), 0.01 (6H,s).

Step 2: Methyl (S)-3-(t-butyldimethylsilyloxy)-2-allyloxypropionate—A stirred solution of methyl (S)-3-(t-butyldimethylsilyloxy)-2-hydroxypropionate (5.84 g) and allyl bromide (10.6 ml) in MeCN (50 ml) at rt was treated portinwise with sodium hydride (1.2 g of a 60% dispersion in oil) over 5 mins. After effervescence ceasde 5% citric acid solution (100 ml) and EtOAc (100 ml) were added. The organic phase was collected washed with water (2×100 ml), saturated brine (100 ml), dried (MgSO₄) and evaporated. The residue was cchromatographed (silica gel, stepgradient 0-10% EtOAc in Light petroleum 40°-60°) to give the subtitle compound (1.2 g). MS APCI (+ion) 275 (MR⁺), ¹H NMR δ(CDCl3) 5.83 (1H.m), 5.16 (2H,m), 4.05 (3H,m), 3.82 (2H,dJ=6.8 Hz), 3.69 (3H,s), 0.83 (9H,s), 0.01 (6H,s).

Step 3: Methyl (S)-3-hydroxy-2-propoxypropionate—A stirred solution of methyl (S)-3-t-butyldimethylsilyloxy)-2-allyloxypropionate (1.2 g), and cyclohexene (5 ml) in MeOH (20 ml) under argon at rt was treated with 10% Pd/C (100 mg). After 72 h the catalyst was filtered off and the solution evaporated. The residue was redissolved in MeOH (10 ml) and 4M HCl in dioxan was added. After 2 h the solution was evaporated to give the subtitled compound. (0.6 g). ¹H NMR δ(CDCl3), 4.02-3.51 (5H,m), 3.77 (3H,s), 2.21 (1H,bs), 1.65 (2H,m), 0.93 (3H,t,J=7.5 Hz).

Step 4: Methyl (R)-3-acetylthio-2-propoxypropionate—An ice-cold solution of triphenylphosphine (1.94 g) in dry THF under argon was treated sropwise with diisopropyl azodicarboxylate (1.46 ml) After 30 mins a solution of methyl (S)-3-hydroxy-2-propoxypropionate (0.6 g) and thioacetic acid (0.53 ml) in dry THF (5 ml) were added dropwise.The mixture was allowed to gain rt overnight and then evaporated. The residue was extracted with hexane (120 ml) and the solution evaporated. The oil was chromatographed (silica gel, stepgradient 5-12% EtOAc in Light petroleum 40°-60°) to give the subtitle compound (0.7 g). ¹H NM (CDCl3), 3.96 (1H,m), 3.77 (3H,s), 3.14-3.60 (4H,m),2.35 (3H,s), 1.65 (2H,m), 0.93 (3H,t,J=7.5 Hz).

Step 4: Methyl (R)-3-(quinolin-3-ylmethanesulfanyl)-2-propoxypropionate—A stiired solution of 3-chloromethylquinoline hydrochloride (0.6 g) and methyl (R)-3-acetylthio-2-propoxypropionate (1.09 g) in MeOH (50 ml) at rt was treated dropwise with 1M NaOH (5.51 ml) and the mixture stirred overnight. The MeOH was evaporated and saturated sodium hydrogen carbonate (10 ml) was added. The aqueous was extracted with MDC (3×10 ml), dried (MgSO₄) and evaporated. The residue was chromatographed (silica gel, stepgradient 10-40% EtOAc in light petroleum 40°-60°) to give the subtitle compound (0.83 g). MS APCI (+ion) 320 ), ¹H NMR δ(CDCl3) 8.90 (1H,d,J=2 Hz), 8.10(2H,m), 7.80 (1H,d,J=6.8 Hz), 7.69 (1H,t,J=6.8 Hz), 7.55 (1H.t,J=6.8 Hz), 4.0 (3H,m), 3.74 (3H,s), 3.31-3.71 (2H,m), 2.76 (2H,m), 1.65 (2H,m), 0.97 (3H,t,J=7.5 Hz).

Step 5: Methyl (R)-3-(quinolin-3-ylmethanesulfoyl)-2-propoxypropionate—A solution of methyl (R)-3-(quinolin-3-ylmethanesulfanyl)-2-propoxypropionate (0.83 g) in MDC (20 ml) was cooled to 0° C. followed by the addition of MCPBA (50%) (2.89 g) and allowed to stir at 0° C. for 30 min. The reaction mixture was quenched with 10% Na₂SO₃ (10 ml) and saturated sodium hydrogen carbonate (10 ml). The MDC layer was dried MgSO₄), evaporated and chromatographed (silica gel, stepgradient 30-60% EtOAc in light petroleum 40°-60°) to give the subtitle compound. (0.6 g). MS APCI (+ion) 352 (MH⁺), ¹H NMR δ(CDCl3) 8.92 (1H,d,J=2 Hz), 8.28(1H,s), 8.13 (1H,d,J=6.8 Hz), 7.85(1H,d,J=6.8 Hz), 7.77 (1H,t,J=6.8 Hz), 7.60 (1H.t,J=6.8 Hz), 4.68-4.48 (3H,m), 3.78(3H,s), 3.90-3.15 (4H,m), 1.83 (2H,m), 1.06 (3H,t,J=7.5 Hz).

Step 6: (R)-3-(Quinolin-3-ylmethanesulfoyl)-2-propoxypropionic acid—A solution of methyl (R)-3-(quinolin-3-ylmethanesulfoyl)-2-propoxypropionate (595 mg) in conc. HCl (10 ml) was refluxed for 30 mins, cooled to rt and evaporated. The residue was re-evaporated from MeOH (10 ml) and DCM (10 ml) to give the subtitled compound. (0.45 g), MS APCI (+ion) 338 (MH⁺), ¹H NMR δ(DMSO) 9.00 (1H,d,J=2 Hz), 8.63 (1H,s), 8.18 (2H,m), 7.95 (1H,t,J=6.8 Hz), 7.77 (1H.t,J=6.8 Hz), 4.86 (2H,Abq),4.34 (1H,m), 3.41-3.71 (4H,m), 1.61 (2H,m), 0.89 (3H,t,J=7.5 Hz).

Step 7: (R)—N-Hydroxy-2-propoxy-3-(quinolin-3-ylmethanesulfonyl)propionamide—A stirred solution of (R)-3-(quinolin-3-ylmethanesulfoyl)-2-propoxypropionic acid (100 mg) in MDC (5 ml) at rt was treated with oxalyl chloride (0.3 ml) and DMF in MDC (1 drop of 10% solution). After 45 mrins the mixture was evaporated and then re-evaporated from MDC (2×5 ml). The residue was suspended in MDC (5 ml) and treated with hydroxylamine hydrochloride (37 mg) and N-methylmorpholine (0.15 ml). After 30 mins water (5 ml) was added and the pH adjusted to 7 (2M HCl). The aqueous was extracted with MDC (5 ml) and the combined MDC layers were dried (MgSO₄) evaporated and chromatographed (acid washed silica gel, stepgradient 0-3% MeOH in MDC) to yield the title compound (17 mg), MS APCI (+ion) 353 (MH⁺), ¹H NMR δ(DMSO), 10.95 (1H,s), 9.09 (1H,s), 8.85 (1H,d,J=2 Hz), 8.38(1H,s), 8.05 (2H,m), 7.82 (1H,t,J=6.8 Hz), 7.65 (1H.t,J=6.8 Hz), 4.80 (2H,Abq),4.20(1H,m), 3.38-3.70 (4H,m), 1.61 (2H,m), 0.91 (3H,t,J=7.5 Hz).

Biological Test Methods

Procedure 1: The ability of test compounds to inhibit the release of soluble CD23 was investigated by use of the following procedure.

RPMI 8866 Cell Membrane CD23 Cleavage Activity Assay:

Plasma membranes from RPMI 8866 cells, a human Epstein-Barr virus transformed B-cell line (Sarfati et al., Immunology 60 [1987] 539-547) expressing high levels of CD23 are purified using an aqueous extraction method. Cells resuspended in homogenization buffer (20 mM HEPES pH 7.4, 150 mM NaCl, 1.5 mM MgCl2, 1 mM DTT) are broken by N₂ cavitation in a Parr bomb and the plasma membrane fraction mixed with other membranes is recovered by centrifugation at 10,000×g. The light pellet is resuspended in 0.2 M potassium phosphate, pH 7.2 using 2 ml per 1-3 g wet cells and the nuclear pellet is discarded. The membranes are further fractionated by partitioning between Dextran 500 (6.4% w/w) and polyethylene glycol (PEG) 5000 (6.4% w/w) (ref), at 0.25 M sucrose in a total of 16 g per 10-15 mg membrane proteins [Morre and Morre, BioTechniques 7, 946-957 (1989)]. The phases are separated by brief centrifugation at 1000×g and the PEG (upper) phase is collected, diluted 3-5 fold with 20 mM potassium phosphate buffer pH 7.4, and centrifuged at 100,000×g to recover membranes in that phase. The pellet is resuspended in phosphate-buffered saline and consists of 3-4 fold enriched plasma membranes as well as some other cell membranes (e.g. lysosomes, Golgi). The membranes are aliquoted and stored at −80° C. Fractionation at 6.6% Dextran/PEG yields plasma membranes enriched 10-fold.

The fractionated membranes are incubated at 37° C. for times up to 4 hrs to produce fragments of CD23 which are separated from the membrane by filtration in 0.2 micron Durapore filter plates (Millipore) after quenching the assay with 5 uM Preparation 1 from WO 95/31457 ([4-(N-Hydroxyamino)-2-(R)-isobutyl-3-(S)-(2-thiophenethiomethyl)succinyl]-(S)-phenylalanine—methylamide sodium salt, prepared according to the procedure descibed in Example 11 of WO 90/05719). sCD23 released from the membrane is determined using the EIA kit from The Binding Site (Birmingham, UK) or a similar one utilising MHM6 anti-CD23 mAb [Rowe et al., Int. J. Cancer, 29, 373-382 (1982)] or another anti-CD23 mAb as the capture antibody in a sandwich EIA. The amount of soluble CD23 made by 0.5 ug membrane protein in a total volume of 50 ul phosphate-buffered saline is measured by EIA and compared to the amount made in the presence of various concentrations of inhibitors. Inhibitors are prepared in solutions of water or dimethylsulfoxide (DMSO) and the final DMSO concentration is not more than 2%. IC50's are determined by curve fitting as the concentration where 50% inhibition of production of sCD23 is observed relative to the difference in sCD23 between controls incubated without inhibitor.

Results

The compound of Example 1 showed an IC₅₀ value of 0.01 μM.

The compound of Example 2 showed an IC₅₀ value of 0.11 μm.

Procedure 2: The ability of test compounds to inhibit matrix metalloproteases was investigated using the following procedures.

Collagenase Inhibition Assay:

The potency of compounds to act as inhibitors of collagenase was determined by the method of Cawston and Barrett (Anal. Biochem. 99, 340-345, 1979), hereby incorporated by reference, whereby a 1 mM solution of the inhibitor being tested or dilutions thereof, was incubated at 37° C. for 18 h with collagen and human recombinant collagenase, from synovial fibroblasts cloned, expressed and purified from E. Coli, (buffered with 150 mM Tris, pH 7.6, containing 15 mM calcium chloride, 0.05% Brij 35, 200 mM sodium chloride and 0.02% sodium azide). The collagen was acetylated ³H type 1 bovine collagen prepared by the method of Cawston and Murphy (methods in Enzymology 80, 711,1981) The samples were centrifuged to sediment undigested collagen and an aliquot of the radioactive supernatant removed for assay on a scintillation counter as a measure of hydrolysis. The collagenase activity in the presence of 1 mM inhibitor, or dilution thereof, was compared to activity in a control devoid of inhibitor and the results reported as that concentration effecting 50% of the collagenase (IC₅₀).

Results

The compound of Example 1 showed an IC₅₀ values of >10 uM.

General MMP Inhibition Assays:

Inhibition of matrix metalloprotease activity was determined using a fluorescence quench assay with appropriate substrate. For example, MMP activity was determined using MMP activated using trypsin, according to Lark et al, Connective Tissue Res. 25, 52 (1990). The MMP is incubated at room temperature in a microtitre plate in a total volume of 100 ul, containing 0.15 M Tris Cl, 15 mM CaCl2, 0.2 M NaCl, pH 7.6 (assay buffer); inhibitor at concentrations up to 100 uM, with no more than 2% DMSO final concentration, 10 uM substrate (such as SDP-3815-PI for MMP-1, Peptides International). The MMP concentration is <10 nM, and determined empirically with the appropriate substrate to give at least a 20-fold increase in fluorescence emission in 30 min. Fluorescent excitation wavelength was 355 nm, emission wavelength 400-460 nm, and data points are collected to generate slope (change in fluorescence with time). Percent inhibition for each concentration is calculated from the slope at time zero, and IC50 values from the concentration dependence. MMP-1, 2, 3, 7, 9, 13, 14 may all be assayed in the same manner, using commercially available substrates reported to be effective for each enzyme. Enzymes were obtained from Calbiochem and activated using the same trypsin method.

Results

The compound of Example 1 showed an IC₅₀ value of >10 uM vs MMP-3.

The compound of Example 2 showed an IC₅₀ value of 7 uM vs MMP-13.

1. A compound of formula (I): 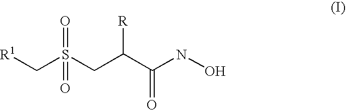
wherein
R is hydrogen, alkyl, alkoxy, alkenyl, alkynyl, aryl, heteroaryl or heterocyclyl; and
R¹ is bicyclyl or heterobicyclyl.

2. A compound of formula (IA):

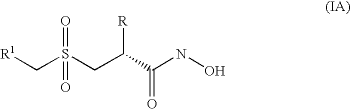

3. A compound according to claim 1, wherein R is aryl or alkoxy and/or R¹ is heterobicyclyl.

4. A compound according claim 1, wherein R is phenyl or propyloxy and/or R¹ is quinoline.

5. A compound selected from:
N-Hydroxy-2-phenyl-3-(3-quinolin-3-ylmethanesulfonyl)-propionamide
(R)—N-Hydroxy-2-propoxy-3-(quinolin-3-ylmethanesulfonyl)-propionamide

6. Use of a compound according to claim 1 for the production of a medicament for the treatment or prophylaxis of disorders in which the overproduction of s-CD23 is implicated.

7. A method for the treatment or prophylaxis of disorders in which the
overproduction of s-CD23 is implicated, which method comprises the administration of a compound according to claim 1 to a human or non-human mammal in need thereof.

8. A pharmaceutical composition for the treatment or prophylaxis of disorders in which the overproduction of s-CD23 is implicated which comprises a compound according to claim 1 and optionally a pharmaceutically acceptable carrier therefor.

9. A process for preparing a compound according to claim 1 which process comprises:
(a) deprotecting a compound of formula (II): 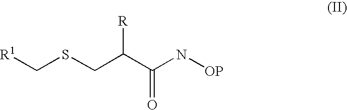
wherein R and R¹ are as defined hereinabove, and P is a protecting group;
(b) oxidising a compound of formula (III): 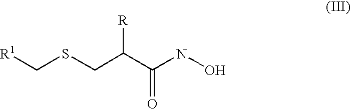
wherein R and R¹ are as defined hereinabove or
(c) converting a compound of formula (I) to a different compound of formula
(I) as defined hereinabove, or
(d) reacting a compound of formula (VIII): 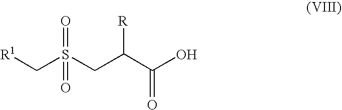
with hydroxylamine or a salt thereof.

10. A compound of formula (II): 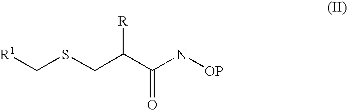
wherein R and R¹ are as defined hereinabove, and P is a protecting group.

11. A compound of formula (III): 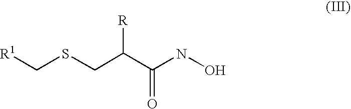
wherein R and R¹ are as defined hereinabove.

12. A compound of formula (VIII): [chemical structure]
